(19) United States
(12) Patent Application Publication
(10) Pub. No.: US 2025/0090442 A1
(43) Pub. Date: Mar. 20, 2025

(54) Topical Composition For Managing Fragile Skin

(71) Applicant: WELMEDIX LLC, Boca Raton, FL (US)

(72) Inventors: Fred Hassan, Boca Raton, FL (US); Sarah Hassan, Boca Raton, FL (US)

(21) Appl. No.: 18/891,842

(22) Filed: Sep. 20, 2024

Related U.S. Application Data
(60) Provisional application No. 63/539,426, filed on Sep. 20, 2023.

(51) Int. Cl. A61K 8/65; A61K 8/42; A61K 8/67; A61K 8/73; A61Q 17/00

(52) U.S. Cl. CPC A61K 8/65; A61K 8/42; A61K 8/671; A61K 8/676; A61K 8/735; A61Q 17/00

(57) ABSTRACT

A topical composition for managing fragile skin includes a moisturizer component including a hyaluronate; an antioxidant component including an ascorbate; a peptide component including collagen peptides; a ceramide component including ceramide; and a retinoid component including retinoid. The % w/w of the collagen peptides is 2.5 to 4 times greater than the % w/w of the ceramide.

### CROSS-REFERENCE TO RELATED APPLICATION

This claims the benefit of priority from U.S. provisional Application No. 63/539,426, filed Sep. 20, 2023, which is incorporated by reference in its entirety.

### FIELD

This relates to the field of skin care products and, more particularly, to skin care products for managing fragile skin.

### BACKGROUND

One of the first visible signs of aging occurs in the skin, which has three main layers: epidermis, dermis, and hypodermis. The epidermis, which is the outermost layer, has the thickness of a sheet of paper and forms a barrier to prevent the body from losing water and pathogens from entering it. The cells in the epidermis are mainly keratinocytes. These specialized skin cells are continually produced at the base of the epidermis. From there, they move toward the skin's surface where they are shed. The middle layer of skin is the dermis, which is about ten times thicker than the epidermis and contains the blood vessels that feed the skin, nerves, sebaceous glands, and hair follicles. The dermis contains collagen fibers that support the skin and elastin fibers that provide flexibility. The hypodermis contains sweat glands, hair follicles, blood vessels, and fat.

As humans age, the skin becomes less elastic because the epidermis and dermis thin and lose collagen and elastin. This gradual atrophy of the epidermis and dermis is a significant cosmetic and medical problem because atrophied skin is more fragile and slower to heal than normal younger healthy skin.

As men and women age or experience certain medical conditions, they naturally lose collagen and a protective fat-intensive layer beneath the skin. After age twenty, collagen production slows and our bodies produce about 1% less collagen in the skin each year. As a result, the skin becomes thinner and more fragile with age.

Fragile skin begins developing in men and women in their 20s and 30s with slowing of collagen production. Collagen production further slows midlife. Particularly in women, collagen loss accelerates in the forties and fifties as estrogen levels start falling during the peri- and post-menopause period. As estrogen levels decline, the skin loses collagen and the protective innermost layer of skin. According to the American Dermatological Association, studies show that the skin loses about 30% of its collagen during the first five years following menopause and that women lose about 2% of their collagen in skin every year for the next 20 years.

This condition in which human skin is fragile and thin is sometimes called “fragile skin” or “thin skin.” Some have also used the term “dermatoporosis” as a technical term for the chronic cutaneous fragility of aging skin. See, Dyer et al., “Chronic Skin Fragility of Aging: Current Concepts in the Pathogenesis, Recognition, and Management of Dermatoporosis,” J Clin Aesthet Dermatol., Vol. 11 (1), pgs. 3-18 2018. Symptoms include skin atrophy, solar purpura, and white pseudoscars on the extremities of patients. Id. They also include skin lacerations and delayed healing of skin. Id. This leaves affected patients susceptible to bleeding and cutaneous infections. Dermatoporosis is predominant on sun-exposed areas such as the forearms and the back of hands. Researchers have developed a method self-diagnosing dermatoporosis. See, Saurat et al, “A simple self-diagnosis tool to assess the prevalence of dermatoporosis in France, “Journal of the European Academy of Dermatology & Venereology,” Vol. 32, pgs. 189-91 (2018). Dermatoporosis is associated with thin, fragile, or “crepey” skin. Other visible symptoms of fragile skin include uneven skin tone, rough skin, itchy skin, flaky skin, redness, and wrinkles.

### BRIEF SUMMARY

A major problem with fragile skin is a weakening of the dermis, which is 80% of the weight of the skin yet presently is not as well targeted by commercial skin care products as the epidermis. A stronger dermis would help the skin remain strong and elastic. A topical composition has been developed to manage fragile skin by targeting the delivery of a combination of skin benefitting compounds to the dermis. The composition is specifically formulated to help penetrate the epidermis toward the dermis to stimulate healing and improve the health of fragile skin and/or proactively try to prevent fragile skin.

An example of a topical composition for managing fragile skin comprises a moisturizer component including 15%-25% w/w hyaluronate; an antioxidant component including 15%-25% w/w ascorbate; a peptide component including 10%-30% w/w collagen peptides; a ceramide component including 3%-20% w/w ceramide; and a retinoid component including 0.1%-10% w/w retinoid. The % w/w of the collagen peptides is 2.5 to 4 times greater than the % w/w of the ceramide.

The topical composition may also include one or more of the following features.

The topical composition may comprise 19%-21% w/w hyaluronate.

The moisturizer component may further include 1%-13% w/w calendula, 0.5%-6% w/w glycerin, 0.1%-4% w/w lanolin, 0.5%-5% w/w aloe vera, and 0.1%-4% w/w dimethicone.

The topical composition may comprise 19%-21% w/w ascorbate.

The antioxidant component may further include 2%-12% w/w vitamin E, 0.5%-5% w/w niacinamide, 0.5%-5% w/w green tea extract, 0.5%-5% w/w curcumin, and 0.5%-5% w/w zinc.

The collagen peptides may be primarily composed of proline, glycine, and hydroxyproline as individual amino acids or oligopeptides with 30 or fewer amino acids.

The topical composition may comprise 18%-22% w/w collagen peptides.

The topical composition may comprise 3%-12% w/w ceramide.

The topical composition may comprise 1%-5% w/w retinoid.

The hyaluronate and the ascorbate may be within 5% w/w of each other.

The retinoid may be water soluble.

An example of a topical product for managing fragile skin comprises a moisturizer component including 0.15%-10% w/w hyaluronate; an antioxidant component including 0.15%-10% w/w of ascorbate; a peptide component including 0.1%-12% w/w collagen peptides; a ceramide component including 0.03%-8% w/w ceramide; and a retinoid component including 0.001%-4% w/w retinoid. The % w/w of the collagen peptides is 2.5 to 4 times greater than the % w/w of the ceramide.

The topical product may also include one or more of the following features.

The topical product may comprise 0.15%-2.5% w/w of the hyaluronate, 0.15%-2.5% w/w ascorbate, 0.1%-3% w/w collagen peptides, 0.03%-2% w/w ceramide, and 0.001%-1% retinoid.

The topical product may comprise 0.15%-2.5% w/w hyaluronate, 0.001%-1.3% w/w calendula, 0.005%-0.6% w/w glycerin, 0.001%-0.4% w/w lanolin, 0.005%-0.5% w/w aloe vera, and 0.001%-0.4% dimethicone.

The topical product may comprise 0.001%-5.2% w/w calendula, 0.005%-2.4% w/w glycerin, 0.001%-1.6% w/w lanolin, 0.005%-2% w/w aloe vera, and 0.001%-1.6% dimethicone.

The topical product may comprise 0.15%-2.5% w/w hyaluronate, 0.15%-2.5% w/w ascorbate, 0.2%-1.2% w/w vitamin E, 0.005%-0.5% w/w niacinamide, 0.005%-0.5% w/w green tea extract, 0.005%-0.5% w/w curcumin, and 0.005%-0.5% w/w zinc.

The topical product may comprise 0.15%-10% w/w ascorbate, 0.2%-4.8% w/w vitamin E, 0.005%-2% w/w niacinamide, 0.005%-2% w/w green tea extract, 0.005%-2% w/w curcumin, and 0.005%-2% w/w zinc.

The collagen peptides may be primarily composed of proline, glycine, and hydroxyproline as individual amino acids or oligopeptides with 30 or fewer amino acids.

The topical product of claim 38, may comprise 0.1%-3% w/w collagen peptides.

The topical product may comprise 0.1%-12% w/w collagen peptides.

The topical product may comprise 0.03%-2% w/w ceramide.

The topical product may comprise 0.03%-8% w/w ceramide.

The topical product may comprise 0.01%-1% w/w retinoid.

The topical product may comprise 0.01%-4% w/w retinoid.

The hyaluronate and the ascorbate may be within 5% w/w of each other.

The retinoid may be water soluble.

An example of a process for preparing a topical product for managing fragile skin comprises diluting a topical composition for managing fragile skin with a cosmetic vehicle suitable for topical application to skin, thereby forming a topical product for managing fragile skin, the topical composition being 1%-40% w/w of the topical product. The topical composition comprises a moisturizer component including 15%-25% w/w hyaluronate; an antioxidant component including 15%-25% w/w ascorbate; a peptide component including 10%-30% w/w collagen peptides; a ceramide component including 3%-20% w/w ceramide; and a retinoid component including 0.1%-10% w/w retinoid. The % w/w of the collagen peptides is 2.5 to 4 times greater than the % w/w ceramide.

The method may also include one or more of the following features.

The cosmetic vehicle may be selected from a skin cleanser, skin lotion, skin ointment, skin serum, and a skin cream.

The cosmetic vehicle may be a skin cleanser and the topical composition may be 1%-10% w/w of the topical product.

The topical composition may comprise 19%-21% w/w hyaluronate.

The moisturizer component may further include 1%-13% w/w calendula, 0.5%-6% w/w glycerin, 0.1%-4% w/w lanolin, 0.5%-5% w/w aloe vera, and 0.1%-4% w/w dimethicone.

The topical composition may comprise 19%-21% w/w ascorbate.

The antioxidant component may further include 2%-12% w/w vitamin E, 0.5%-5% w/w niacinamide, 0.5%-5% w/w green tea extract, 0.5%-5% w/w curcumin, and 0.5%-5% w/w zinc.

The collagen peptides may be primarily composed of proline, glycine, and hydroxyproline as individual amino acids or oligopeptides with 30 or fewer amino acids.

The topical composition may comprise 18%-22% w/w collagen peptides.

The topical composition may comprise 3%-12% w/w ceramides.

The topical composition may comprise 1%-5% w/w retinoid.

The hyaluronate and the ascorbate may be within 5% w/w of each other.

The retinoid may be water soluble.

An example of a method of managing dermatoporosis comprises applying to skin having dermatoporosis a topical product comprising a hyaluronate, an ascorbate, collagen peptides, a ceramide, and a retinoid. A % w/w of the collagen peptides is 2.5 to 4 times greater than a % w/w of the ceramide.

The method may also include one or more of the following features.

The topical product may comprise 0.15%-2.5% w/w of the hyaluronate, 0.15%-2.5% w/w of the ascorbate, 0.1%-3% w/w collagen peptides, 0.03%-2% w/w of the ceramide, and 0.001%-1% of the retinoid.

The topical product may comprise 0.15%-10% w/w of the hyaluronate, 0.15%-10% w/w of the ascorbate, 0.1%-12% w/w collagen peptides, 0.03%-8% w/w ceramides, and 0.001%-4% retinoid.

The topical product may comprise 0.15%-2.5% w/w of the hyaluronate, 0.001%-1.3% w/w calendula, 0.005%-0.6% w/w glycerin, 0.001%-0.4% w/w lanolin, 0.005%-0.5% w/w aloe vera, and 0.001%-0.4% dimethicone.

The topical product may comprise 0.15%-10% w/w of the hyaluronate, 0.001%-5.2% w/w calendula, 0.005%-2.4% w/w glycerin, 0.001%-1.6% w/w lanolin, 0.005%-2% w/w aloe vera, and 0.001%-1.6% dimethicone.

The topical product may comprise 0.15%-2.5% w/w of the hyaluronate, 0.15%-2.5% w/w of the ascorbate, 0.2%-1.2% w/w vitamin E, 0.005%-0.5% w/w niacinamide, 0.005%-0.5% w/w green tea extract, 0.005%-0.5% w/w curcumin, and 0.005%-0.5% w/w zinc.

The topical product may comprise 0.15%-10% w/w of the hyaluronate, 0.15%-10% w/w of the ascorbate, 0.2%-4.8% w/w vitamin E, 0.005%-2% w/w niacinamide, 0.005%-2% w/w green tea extract, 0.005%-2% w/w curcumin, and 0.005%-2% w/w zinc.

The collagen peptides may be primarily composed of proline, glycine, and hydroxyproline as individual amino acids or oligopeptides with 30 or fewer amino acids.

The topical product may comprise 0.1%-3% w/w of the collagen peptides.

The topical product may comprise 0.1%-12% w/w of the collagen peptides.

The topical product may comprise 0.03%-2% w/w of the ceramides.

The topical product may comprise 0.03%-8% w/w of the ceramides.

The topical product may comprise 0.01%-1% w/w of the retinoid.

The topical product may comprise 0.01%-4% w/w of the retinoid.

The hyaluronate and the ascorbate may be within 5% w/w of each other.

The retinoid may be water soluble.

Any feature associated with any of the above-mentioned compositions and methods may be combined with any of the other compositions and methods.

### BRIEF DESCRIPTION OF THE DRAWINGS

FIG. 1 is a diagram illustrating cornerstones of the of the topical composition.

FIG. 2 is a diagram of skin layers.

### DETAILED DESCRIPTION

This disclosure describes examples, but not all possible examples and features of the compositions and methods. Where a particular feature is disclosed in the context of a particular example, that feature can also be used, to the extent possible, in combination with and/or in the context of other examples. The compositions and methods may be embodied in many different forms and should not be construed as limited to only the examples described here.

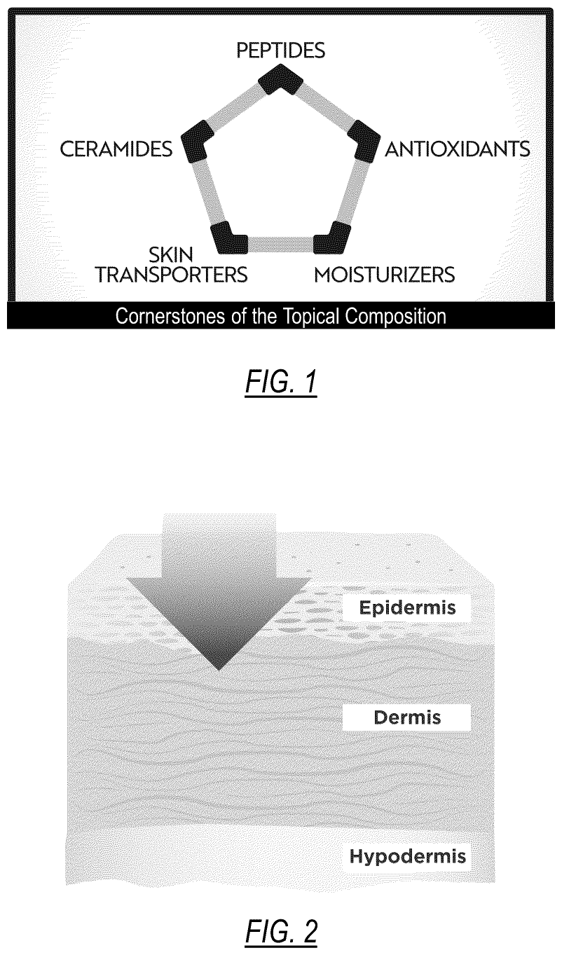

Referring to FIG. 1, the topical composition includes a moisturizer component, an antioxidant component, a peptide component, a ceramide component, and a skin transporter component, which are the five cornerstone components of the topical composition. These components are specifically selected and combined so that they can help toward penetrating the epidermis toward the dermis. Each of these five components represent five cornerstones to managing fragile skin as they are designed to work together to moisturize, heal, restore, and strengthen the protective skin barrier through to the dermis layer, which has been weakened and damaged over time by being affected with fragile skin.

Referring to FIG. 2, the topical composition is formulated to manage fragile skin by transporting the components through the epidermis toward the dermis. This is different than conventional skin care products, which typically remain on the surface of the epidermis or the outer layers of the epidermis, primarily the stratum corneum.

Fragile skin is characterized by thinning, more translucent skin, a loss of skin strength, and the characteristics described above. This skin degradation process is caused by a loss of skin proteins, specifically collagen peptides essential to the production of collagen, a loss of ceramides, a loss of moisture as the body produces less hyaluronic acid, and exposure to free radicals over time (e.g., environmental pollutants, stress, UV rays) that damage skin cells. Fragile skin is more common in older adults, but it can occur at any age due to sun exposure, genetics, autoimmune diseases (e.g., diabetes) and certain medications. Fragile skin is more prone to drying, irritation, infection, bruising, pressure sores, and skin tears. Serious cases of fragile skin can lead to hospitalization. Anyone can have fragile skin at any age. As used herein the term “fragile skin” includes thin skin, crepey skin, and skin affected by dermatoporosis.

The topical composition includes a plurality of skin benefitting ingredients that are specifically selected to work together to manage fragile skin. The composition is intended to deliver the skin benefitting ingredients towards the dermis for promoting skin repair and regeneration, strengthening fragile skin, stimulating collagen production, and improving the overall health of the affected skin. The topical composition may be used to prevent or delay further weakening of skin and/or to improve the function of fragile skin. The topical composition is specifically formulated to help make the beneficial skin ingredients penetrate the epidermis to stimulate healing and improve the health of affected skin. Since there is a broad range of skin types, by design, the range of formulations disclosed herein is broad.

The topical composition may have a concentrate form and a dilute form. The concentrate form has a high concentration of skin benefitting ingredients falling within the five cornerstone components mentioned above.

Table 1 provides several examples of the concentrate form of the topical composition. The % represents the percent by weight (% w/w) of each component relative to the total weight of the topical composition.

TABLE 1
Certain examples of the topical composition

| Component | Example A | Example B | Example C | Example D |
|---|---|---|---|---|
| Antioxidant | 35% | 30%-40% | 32%-38% | 34%-36% |
| component | | | | |
| Moisturizer | 35% | 30%-40% | 32%-38% | 34%-36% |
| component | | | | |
| Peptide | 20% | 10%-30% | 12%-28% | 18%-22% |
| component | | | | |
| Ceramide | 7% | 3%-20% | 3%-12% | 5%-9% |
| component | | | | |
| Skin | 3% | 0.1%-10% | 1%-5% | 2%-4% |
| transporter | | | | |
| component | | | | |

The balance of the % w/w of the topical composition is a dermatologically acceptable solvent and dermatologically acceptable excipients. In most cases, the balance is all or mostly water.

Examples of the moisturizer component, antioxidant component, peptide component, ceramide component, and a skin transporter component are now described in more detail.

### Skin Transporter Component

The skin transporter component is designed to perform two functions; namely, it benefits fragile skin and may help transport other skin benefitting ingredients in the topical composition into the epidermis towards the dermis.

The skin transporter component includes at least one retinoid. Retinoids are particularly useful because they benefit fragile skin and may enhance the penetration of some of the other beneficial ingredients of the topical composition into the skin. The retinold may function as a skin penetration enhancer for certain components of the topical composition.

When a skin penetration enhancing retinoid is combined with small molecule versions of other ingredients, the retinoid may help the other components penetrate towards the dermis.

Some retinoids are known irritate the skin when applied at relatively high concentrations. When managing fragile skin, the retinold and its concentration should be such that the retinoid does not cause further damage or discomfort to the skin.

Vitamin A (retinol) and other related retinoid compounds, Including retinoic acid, have been used in topical skin-care products having both cosmetic and therapeutic benefits. Retinol and retinoic acid have been used to treat acne, for example. Retinoids also can reverse the skin ageing process caused by increasing age and external factors such as sun exposure.

Examples of retinoids that may be used include retinol, retinoic acids (including trans-retinoic acids), and derivatives thereof. Such derivatives may include esters, amides, ethers, and retinal. Retinoids may also include solvates including hydrates as well as the corresponding salts thereof, where applicable, such as alkali metal salts and alkaline earth metal salts.

In certain examples of the retinoid component, the retinoid includes one or more retinol esters. Retinol esters may be beneficial for fragile skin because they tend to cause less skin irritation and function as a skin penetration enhancer for the other components of the topical composition.

Examples of retinol esters (or retinyl esters) include C₂₋₂₀ alkanoyl esters of retinol. In some cases, these C₂₋₂₀ alkanoyl esters may be derived from C₃₋₂₀ fatty acids, from propionic acid to arachidic acid. Examples of retinol esters (retinyl esters) include, for example retinyl acetate, retinyl propionate, retinyl butyrate, retinyl valerate, retinyl caproate, retinyl palmitate, and the like.

As used herein, the term “retinoid” includes a single retinoid or multiple retinoids.

### Antioxidant Component

The antioxidant component is a combination of antioxidant ingredients that are beneficial to the skin and provide protection against harmful free radicals. Antioxidants may also facilitate collagen biosynthesis and are beneficial towards healthy skin.

Table 2 provides several examples of the antioxidant component of the topical composition. In Table 2, the % represents the percent by weight (% w/w) of each component relative to the total weight of the topical composition.

TABLE 2
Certain examples of the antioxidant component

| Ingredient | Example A | Example B | Example C | Example D |
|---|---|---|---|---|
| Ascorbate | 20% | 15%-25% | 17%-23% | 19%-21% |
| Vitamin E | 7% | 2%-12% | 5%-10% | 6%-8% |
| Niacinamide | 2% | 0.5%-5% | 0.5%-4% | 1%-3% |
| Green Tea | 2% | 0.5%-5% | 0.5%-4% | 1%-3% |
| Extract | | | | |
| Curcumin | 2% | 0.5%-5% | 0.5%-4% | 1%-3% |
| Zinc | 2% | 0.5%-5% | 0.5%-4% | 1%-3% |

In Example A, the total % w/w of the antioxidant component ingredients is 35%, which corresponds to Example A of Table 1.

The primary antioxidant in the antioxidant component is an ascorbate. Examples of ascorbates include ascorbic acid and/or derivatives thereof, including ascorbic acid. Ascorbic acid, also called vitamin C, is an organic carboxylic acid, which is usually in the L-isomer form (L-ascorbic acid). Ascorbic acid is a powerful antioxidant.

Ascorbates used in the topical composition are ascorbic acid and/or derivatives thereof. These derivatives include ascorbates, ascorbate salts, ascorbate esters, and ascorbate sugars. More particular examples of ascorbic acid derivatives include, but are not limited to, sodium ascorbate, calcium ascorbate, potassium ascorbate, magnesium ascorbate, magnesium ascorbylphosphate, sodium ascorbyphosphate. Other examples include ascorbyl esters, such as ascorbyl acetate, ascorbyl propionate, ascorbyl palmitate, and ascorbyl stearate.

The ascorbate may, if desired, be in a stabilized form that is less prone to oxidation than other ascorbates.

The ascorbate may help increase collagen production and, when combined with a skin penetration enhancing retinoid, may be able to penetrate toward the dermis more readily than the ascorbate in the absence of the retinoid.

As used herein, the term “ascorbate” includes a single ascorbate compound or multiple ascorbate compounds.

The retinoid may promote a water soluble ascorbate to penetrate deeper into the skin towards the dermis. Given this, it may be advantageous in certain examples of the topical composition to select a water soluble ascorbate to use in the antioxidant component.

In other examples of the antioxidant component, the ascorbate is lipid soluble to promote transport toward the dermis after topical application to the skin. Ascorbyl palmitate is one example of a lipid soluble ascorbate. When combined with a skin penetration enhancing retinoid, a lipid soluble ascorbate may be able to penetrate the epidermis more readily than the ascorbate on its own.

The mass transfer of the ascorbate may occur via an aqueous pathway, lipid pathway, or both. In certain examples of the antioxidant component, calcium ascorbate is used because it is a mass transfer facilitator via the aqueous pathway. In other examples of the antioxidant component, ascorbyl palmitate ester is used to facilitate extra mass transfer via the lipid pathway.

### Moisturizer Component

The moisturizer component is a combination of skin moisturizing ingredients. Table 3 provides several examples of the moisturizer component. In Table 3, the % represents the percent by weight (% w/w) of each component relative to the total weight of the topical composition.

TABLE 3
Certain examples of the moisturizer component

| Ingredient | Example A | Example B | Example C | Example D |
|---|---|---|---|---|
| Hyaluronate | 20% | 15%-25% | 17%-23% | 19%-21% |
| Calendula | 8% | 1%-13% | 4%-11% | 7%-9% |
| Glycerin | 3% | 0.5%-6% | 1%-5% | 2%-4% |
| Lanolin | 1% | 0.1%-4% | 0.1%-3% | 0.1%-2% |
| Aloe Vera | 2% | 0.5%-5% | 0.5%-4% | 1%-3% |
| Dimethicone | 1% | 0.1%-4% | 0.1%-3% | 0.1%-2% |

In Example A, the total % w/w of the moisturizer component ingredients is 35%, which corresponds to Example A of Table 1.

The primary moisturizer component ingredient is a hyaluronate. Hyaluronates are primary components of the extracellular matrix and are found in significant quantities in normal, healthy skin.

Hyaluronates are disaccharides composed of D-glucuronic acid and N-acetylglucosamine units linked together with glucosidic bonds. Hyaluronates include hyaluronic acid, a hydrolyzed form thereof, hyaluronate salts, and the like. Certain examples of hyaluronate salts include sodium hyaluronate, potassium hyaluronate, calcium hyaluronate, and magnesium hyaluronate.

As used herein, the term “hyaluronate” includes a single hyaluronate compound or multiple hyaluronate compound.

The skin penetration enhancing retinoid may improve the skin penetration of the hyaluronate, allowing the hyaluronate to move toward the dermis. In some examples it is desirable to use a soluble hyaluronate, such as a water soluble or lipid soluble hyaluronate. This is because hyaluronates are typically large molecules that cannot penetrate the epidermis. Using a soluble hyaluronate in the topical composition may be beneficial because the retinoid will enhance penetration of the soluble hyaluronate toward the dermis. In some examples, the soluble hyaluronate is a water soluble hyaluronate salt such as sodium hyaluronate or the like.

### Peptide Component

About 70% of the dermis is composed of collagen. Collagen gives the dermis its flexibility and strength. To assist the dermis with producing collagen, the topical composition includes collagen peptides. The collagen peptides may be present as individual molecules, dipeptides, tripeptides, oligopeptides, polypeptides, or polypeptide collagen fragments.

In certain examples of the collagen peptide component, the collagen peptides selected are relatively small molecules to assist with penetration through the epidermis. Such collagen peptides may be individual amino acids and/or as oligopeptides with thirty or fewer amino acids.

In a particular example of the topical composition, the collagen peptides are primarily proline, glycine, and hydroxyproline. These amino acids are found naturally in the dermis and represent about 57% of the total amino acids present in collagen. As individual molecules, they can penetrate the epidermis because their average molecular weight is only about 101 Da, with glycine being 75 Da, proline being 97.15 Da, and hydroxyproline being 131.13 Da. These peptides may increase skin elasticity, increase collagen and elastin production, and promote skin repair and regeneration. They may also play a key role in improving the skin's texture and strength.

Other peptides, natural or artificial may also be included in the topical composition. Examples of natural peptides include, but are not limited to, phenylalanine, lysine, tryptophan, leucine, methionine, threonine, tyrosine, arginine, histidine, isoleucine, glutamine, alanine, glycine, proline, cysteine, asparagine, valine, and serine.

### Ceramide Component

Ceramides are lipids found primarily in the outermost layer of the epidermis, called the stratum corneum. Ceramides play a role in protecting the skin's barrier function against damage and in maintaining skin hydration. Ceramides represent about 30 to 40% of the lipids that are present in the skin.

Ceramides, which are composed of a sphingosine and a fatty acid, are categorized using the following nomenclature: a-hydroxy fatty acid (A), w-hydroxy fatty acid (O), non-hydroxy fatty acid (N), ester-linked non-hydroxy fatty acid (E), sphingosine(S), phytosphingosine (P), dihydrosphingosine (DS), and 6-hydroxy-sphingosine moieties (H).

These are further subdivided into types of ceramides: ceramide NDS, ceramide NS, ceramide NP, ceramide NH, ceramide ADS, ceramide AS, ceramide AP, ceramide AH, ceramide EODS, ceramide EOS, ceramide EOP, and ceramide EOH. Ceramides may also include caprooyl phytosphingosine and caprooyl sphingosine.

A particular example of the ceramide component includes seven ceramide compounds, which are ceramide NP, ceramide NS, ceramide EOS, ceramide EOP, ceramide AP, caprooyl phytosphingosine, and caprooyl sphingosine.

As used herein, the term “ceramide” includes a single ceramide compound or multiple ceramide compounds. Ceramide may, for example, include one or more of the aforementioned ceramide compounds and/or any other ceramide compound or combination of different ceramide compounds.

Most topical skin care products containing ceramides are formulated so that the ceramides remain at or near the surface of the epidermis. The topical composition described here, however, is formulated in such a way as to help promote the ceramides to penetrate the epidermis toward the dermis. Using a retinoid that functions as a skin penetration enhancer may further help the ceramides penetrate the epidermis toward the dermis.

Some of the ingredients described herein as being associated with a particular component may have more than one function. For example, ascorbate may be in the antioxidant component, but it also can have moisturizer properties. As another example, a ceramide may also be a moisturizer in addition to being a beneficial skin lipid.

### Important Relative Concentrations

Although the % w/w of different skin benefitting ingredients can vary between different examples of the topical composition, the relative amounts of certain skin benefitting ingredients may be confined within desired limits.

The approximate ratio of collagen peptides vs. ceramides in the skin is 3:1. Ceramides represent about 30 to 40% of the lipids that are present in the skin and about 70% of the dermis is composed of collagen. This ratio also approximates the thickness of the dermis vs the epidermis. It may be advantageous in some examples of the topical composition, for the concentration of collagen peptide component is about 3, about 3.5, or 2.5 to 4 times greater than the concentration of the ceramide component to approximate the 3:1 ratio In some examples of the topical composition, the ratio of the collagen peptide component to the ceramide component is about 3:1.

In some examples of the topical composition, the % w/w of the moisturizer component and antioxidant component may be equivalent or plus or minus 5% w/w of each other. This is because the ceramides also have moisturizing properties.

In some examples of the topical composition, the % w/w of the hyaluronate and the ascorbate are within 5% w/w of each other.

In some examples of the topical composition, the retinoid component has the lowest % w/w of all the components.

In some examples of the topical composition, it may be desirable for most of the ingredients in the topical composition to have a molecular weight of 500 Da or less. This relatively low molecular weight promotes skin penetration towards the dermis.

In some examples of the topical composition, the ascorbate, retinoid, collagen peptides, and ceramide have a molecular weight of 500 Da or less. The retinoid, as a skin penetration enhancer, may enhance penetration of these ingredients toward the dermis.

### Preparing the Topical Product

The topical composition may be prepared by combining the ingredients therein and mixing them. Each component may be prepared separately and then mixed with the other components' ingredients or all the ingredients may be combined and mixed at the same time.

If needed to aid the mixing process, the topical composition may also include one or more solvents, surfactants, emulsifiers, and the like. Heating and/or cooling may be applied during the preparation process. In a typical example, most or all of the remaining % w/w balance of the topical composition is water.

Mixing may include techniques such as stirring, blending, dispersing, milling, homogenizing, and the like. The mixing of the components may form a solution or may not form a solution. The mixture may form a colloidal suspension.

### Dilution of the Concentrate Version of the Topical Composition to Form a Topical Product

The concentrate form of the topical composition may be diluted to form a topical product for managing fragile skin. A process for preparing the topical product for managing fragile skin includes diluting the topical composition for managing fragile skin with a cosmetic vehicle suitable for topical application to skin, thereby forming the topical product for managing fragile skin.

The concentrate form of the topical composition may be stored in a container and subsequently combined with the cosmetic vehicle, which is dermatologically acceptable for application to human skin. Examples of cosmetic vehicles include emulsions (oil-in-water and water-in-oil), gels, solids, creams, liquids, lotions, ointments, serums, sprays, foams, deodorants, pre-moistened wipes and washcloths, and the like.

In particular examples, the cosmetic vehicle is a skin cleanser, skin lotion, skin ointment, skin serum, and/or skin cream. These may be conventional skin cleansers, skin lotions, skin ointments, skin serums, and/or skin creams with conventional ingredients for each.

The resulting topical product made by diluting the topical composition with the cosmetic vehicle may be applied directly to human skin for the management of fragile skin. The topical product may take the form of the cosmetic vehicle used. For example, if the cosmetic vehicle is a skin cleanser, skin lotion, skin ointment, skin serum, and/or skin cream, the topical product is a corresponding skin cleanser, skin lotion, skin ointment, skin serum, and/or skin cream containing the diluted topical product. Other examples of cosmetic vehicles and corresponding topical products include cosmetics, sunscreens, foundations, night creams, toners, masks, antiaging products, and the like. In this case, the ingredients in the topical composition dissolve or disperse within the topical product, giving the topical product the therapeutic benefits of the topical composition.

The concentrate form of the topical composition can be used to make topical products that address a variety of skin conditions that can be made worse or more vulnerable by fragile skin such as itchy skin, dry skin, contact dermatitis, bruising, rough or bumpy skin, chafing, foot related skin conditions including fungal infections, psoriasis, eczema, wrinkles, saggy or crepey skin, age spots, rosacea, heat rash, hives, diaper rash, sunscreens, wound care, incontinence care. Certain examples of the topical product containing the topical composition may also be gentle enough to be used in baby skin products.

The topical composition may be diluted with the cosmetic vehicle by mixing the pertinent ingredients together using a conventional mixing process.

In some examples, the topical product and topical composition are prepared separately and later mixed. In other examples, the topical composition and topical product are prepared simultaneously as if the topical composition is an integral part of the topical product.

The topical product may also include one or more of the skin benefiting ingredients aside from the skin benefitting ingredients from the topical composition.

Certain examples of the topical product may include, for example, petrolatum, aloe vera, coconut oil, calendula oil, soybean oil, zinc oxide, shea butter, dimethicone, and/or white birch bark extract.

The topical product is dermatologically acceptable in that it is substantially non-toxic and non-irritating when applied to skin, including fragile skin. The topical product may be formulated to have a desired viscosity for appropriate application to the skin.

When the concentrate form of the topical composition is diluted with the cosmetic vehicle to form the topical product, the % w/w of the composition with respect to the total weight of the topical product may be 1% w/w to 40% w/w, 1% w/w to 35% w/w, 1% w/w to 10% w/w, 2% w/w to 8% w/w, 3% w/w to 6% w/w, about 5% w/w, 10% w/w to 40% w/w, 20% w/w to 40% w/w, 25% w/w to 35% w/w, or about 30% w/w.

In some examples, the topical product is left on the skin after application. Such examples include skin lotions, skin creams, skin ointments, skin serums, or the like. In these examples the % w/w of the topical composition with respect to the total weight of the topical product may be, for example 1% w/w to 40% w/w, 1% w/w to 35% w/w, 10% w/w to 40% w/w, 20% w/w to 40% w/w, 25% w/w to 35% w/w, or about 30% w/w.

In some examples, the topical product is washed off the skin after application. Such examples include skin cleansers and the like. In these examples the % w/w of the composition with respect to the total weight of the topical product may be, for example 1% w/w to 10% w/w, 2% w/w to 8% w/w, 3% w/w to 6% w/w, or about 5% w/w.

The % w/w of an ingredient in the topical product may be calculated by multiplying the % w/w of the topical composition ingredient with the % w/w of the topical composition with respect to the total weight of the topical product using the equation shown below.

% w/w ingredient in the topical product=% w/w ingredient in the topical composition*% w/w of topical product with respect to total weight of the topical composition

Table 4 provides results using this equation on an example of the topical composition. This table converts the % w/w of each ingredient into its concentration in the topical product.

TABLE 4
Concentration of ingredients in the topical product

| | If topical product | | If topical product | |
|---|---|---|---|---|
| Ingredient % w/w | is 1%-10% w/w | | is 1%-40% w/w | |
| of topical composition | topical composition | | topical composition | |
| 15%-25% w/w | 0.15%-2.5% | w/w | 0.15%-10% | w/w |
| hyaluronate | | | | |
| 15%-25% w/w | 0.15%-2.5% | w/w | 0.15%-10% | w/w |
| ascorbate | | | | |
| 10%-30% w/w collagen | 0.1%-3% | w/w | 0.1%-12% | w/w |
| peptides | | | | |
| 3%-20% w/w ceramide | 0.03%-2% | w/w | 0.03%-8% | w/w |
| 0.1%-10% w/w retinoid | 0.001%-1% | w/w | 0.001%-4% | w/w |

If the topical composition is 1%-10% w/w of the topical product, the % w/w of the moisturizer component ingredients corresponding to Example B of Table 3 is 0.15%-2.5% w/w of the hyaluronate, 0.001%-1.3% w/w calendula, 0.005%-0.6% w/w glycerin, 0.001%-0.4% w/w lanolin, 0.005%-0.5% w/w aloe vera, and 0.001%-0.4% dimethicone.

If the topical composition is 1%-40% w/w of the topical product, the % w/w of the moisturizer component ingredients corresponding to Example B of Table 3 is 0.15%-10% w/w of the hyaluronate, 0.001%-5.2% w/w calendula, 0.005%-2.4% w/w glycerin, 0.001%-1.6% w/w lanolin, 0.005%-2% w/w aloe vera, and 0.001%-1.6% dimethicone.

If the topical composition is 1%-10% w/w of the topical product, the % w/w of the antioxidant component ingredients corresponding to Example B of Table 2 is 0.15%-2.5% w/w of the ascorbate, 0.2%-1.2% w/w vitamin E, 0.005%-0.5% w/w niacinamide, 0.005%-0.5% w/w green tea extract, 0.005%-0.5% w/w curcumin, and 0.005%-0.5% w/w zinc.

If the topical composition is 1%-40% w/w of the topical product, the % w/w of the antioxidant component ingredients corresponding to Example B of Table 2 is 0.15%-10% w/w of the hyaluronate, 0.15%-10% w/w of the ascorbate, 0.2%-4.8% w/w vitamin E, 0.005%-2% w/w niacinamide, 0.005%-2% w/w green tea extract, 0.005%-2% w/w curcumin, and 0.005%-2% w/w zinc.

### Method of Using the Topical Composition and Product

The topical composition and topical product may be used in a method of managing fragile skin. The term “managing fragile skin” means at least one of: treating fragile skin, improving the health of fragile skin, improving the appearance of fragile skin, reducing the symptoms of fragile skin, proactively reducing the onset of fragile skin, and/or treating dermatoporosis.

The fragile skin being managed may have many different causes, including skin atrophy due to age, sun exposure, radiation damage, chemotherapy, autoimmune diseases, heredity, free radical damage, chemical damage, physical damage, or another cause.

The method includes topically applying the topical composition or topical product to person's skin in need thereof. The topical composition or topical product may be applied sporadically, daily, or multiple times a day, depending on the quality of the person's skin and how the person's skin responds. The topical composition or topical product may be advantageous when applied daily and proactively to manage fragile skin.

The topical composition or topical product may be used when fragile skin is not present to improve the appearance of skin, strengthen skin, improve skin elasticity, moisturize the skin, and to prevent or delay the onset of fragile skin, including for use on diaper rash and/or pressure sores, for example.

The topical composition may proactively improve fragile skin through the unique combination of ingredients designed to penetrate the epidermis and target the dermis. In some people, the topical composition may firm or strengthen the skin, increase the skin's elasticity, reduce discoloration, reduce the appearance of crepey skin, and reduce signs aging among other benefits.

The topical composition may be applied to any type of skin in need of fragile skin management, such as the face, neck, arms, hands, legs, feet, buttocks, perianal area, and other areas.

As used herein, topical application and variations thereof refers to applying and/or spreading a composition onto the surface of the skin. Topical application is achieved differently for different types of topical products. Topical products that are designed to be left on the skin for long periods are typically sprayed or rubbed onto the skin. Topical products that are designed to be washed off the skin, such as cleansers, may or may not be combined with water while cleansing the skin.

A particular method of use of the topical composition is a method of managing dermatoporosis. The method includes applying to skin having dermatoporosis a topical product comprising a hyaluronate, an ascorbate, collagen peptides, ceramides, and a retinoid. In this topical product the % w/w of the collagen peptides is 2.5 to 4 times greater than the % w/w of the ceramides. This topical product may be a diluted form of the topical composition using a cosmetic vehicle described above.

In an example of the method, the topical composition is 1%-10% w/w of the total weight of the topical product and the topical product includes 0.15%-2.5% w/w of the hyaluronate, 0.15%-2.5% w/w of the ascorbate, 0.1%-3% w/w collagen peptides, 0.03%-2% w/w ceramide, and 0.001%-1% retinoid.

In an example of the method, the topical composition is 1%-40% w/w of the total weight of the topical product and the topical product includes 0.15%-10% w/w of the hyaluronate; 0.15%-10% w/w of the ascorbate, 0.1%-12% w/w collagen peptides, 0.03%-8% w/w ceramide, and 0.001%-4% retinoid.

In an example of the method, the topical composition is 1%-10% w/w of the total weight of the topical product and, in the topical product, the moisturizer component is 0.15%-2.5% w/w of the hyaluronate, 0.001%-1.3% w/w calendula, 0.005%-0.6% w/w glycerin, 0.001%-0.4% w/w lanolin, 0.005%-0.5% w/w aloe vera, and 0.001%-0.4% dimethicone.

In an example of the method, the topical composition is 1%-10% w/w of the total weight of the topical product and, in the topical product, the moisturizer component is 0.15%-10% w/w of the hyaluronate, 0.001%-5.2% w/w calendula, 0.005%-2.4% w/w glycerin, 0.001%-1.6% w/w lanolin, 0.005%-2% w/w aloe vera, and 0.001%-1.6% dimethicone.

In an example of the method, the topical composition is 1%-10% w/w of the total weight of the topical product and, in the topical product, the antioxidant component is 0.15%-2.5% w/w of the hyaluronate, 0.15%-2.5% w/w of the ascorbate, 0.2%-1.2% w/w vitamin E, 0.005%-0.5% w/w niacinamide, 0.005%-0.5% w/w green tea extract, 0.005%-0.5% w/w curcumin, and 0.005%-0.5% w/w zinc.

In an example of the method, the topical composition is 1%-40% w/w of the total weight of the topical product and, in the topical product, the antioxidant component is 0.15%-10% w/w of the hyaluronate, 0.15%-10% w/w of the ascorbate, 0.2%-4.8% w/w vitamin E, 0.005%-2% w/w niacinamide, 0.005%-2% w/w green tea extract, 0.005%-2% w/w curcumin, and 0.005%-2% w/w zinc.

In an example of the method, the topical composition is 1%-10% w/w of the total weight of the topical product and the topical product includes 0.1%-3% w/w collagen peptides.

In an example of the method, the topical composition is 1%-40% w/w of the total weight of the topical product and the topical product 0.1%-12% w/w collagen peptides.

In an example of the method, the topical composition is 1%-10% w/w of the total weight of the topical product and the topical product includes 0.03%-2% w/w ceramide.

In an example of the method, the topical composition is 1%-40% w/w of the total weight of the topical product and the topical product includes 0.03%-8% w/w ceramide.

In an example of the method, the topical composition is 1%-10% w/w of the total weight of the topical product and the topical product includes 0.01%-1% w/w retinoid.

In an example of the method, the topical composition is 1%-40% w/w of the total weight of the topical product and the topical product includes 0.01%-4% w/w retinoid.

### EXAMPLE

This example is provided to provide details of a particular example of the topical composition and a topical product and does not limit the scope of possible examples.

An example of the topical composition is prepared by mixing the following ingredients in an aqueous solution.

Antioxidant component: 20% w/w calcium ascorbate
7% w/w vitamin E
2% w/w niacinamide
2% w/w green tea extract
2% w/w curcumin
2% w/w zinc
   Moisturizer component: 20% w/w sodium hyaluronate
8% w/w calendula
3% w/w glycerin
1% w/w lanolin
2% w/w aloe vera
1% w/w dimethicone
   Peptide component: 20% w/w collagen peptides
   Ceramide component: 7% ceramides
   Skin transporter component: 3% retinol

The % w/w of these ingredients is the % w/w of each ingredient with respect to the total weight of the topical composition.

A skin cream containing the topical composition is prepared by obtaining a pre-existing dermatologically acceptable skin cream and adding the topical composition to it until the skin cream is 2% w/w of topical composition. The concentration of the topical composition ingredients with respect to the total weight of the skin cream, therefore, is 2% of the concentrations listed above.

This skin cream containing the topical composition is used to manage fragile skin by rubbing the skin cream containing the topical composition onto the fragile skin daily.

The compositions and methods are not limited to the details described in connection with the example embodiments. There are numerous variations and modification of the compositions and methods that may be made without departing from the scope of what is claimed.

We claim:

1. A topical composition for managing fragile skin, the topical composition comprising:
a moisturizer component including 15%-25% w/w hyaluronate;
an antioxidant component including 15%-25% w/w ascorbate;
a peptide component including 10%-30% w/w collagen peptides;
a ceramide component including 3%-20% w/w ceramide; and
a retinoid component including 0.1%-10% w/w retinoid;
wherein the % w/w of the collagen peptides is 2.5 to 4 times greater than the % w/w of the ceramide.

2. The topical composition of claim 1, comprising 19%-21% w/w hyaluronate.

3. The topical composition of claim 1, wherein the moisturizer component further includes 1%-13% w/w calendula, 0.5%-6% w/w glycerin, 0.1%-4% w/w lanolin, 0.5%-5% w/w aloe vera, and 0.1%-4% w/w dimethicone.

4. The topical composition of claim 1, comprising 19%-21% w/w ascorbate.

5. The topical composition of claim 1, wherein the antioxidant component further includes 2%-12% w/w vitamin E, 0.5%-5% w/w niacinamide, 0.5%-5% w/w green tea extract, 0.5%-5% w/w curcumin, and 0.5%-5% w/w zinc.

6. The topical composition of claim 1, wherein the collagen peptides are primarily composed of proline, glycine, and hydroxyproline as individual amino acids or oligopeptides with 30 or fewer amino acids.

7. The topical composition of claim 1, comprising 18%-22% w/w collagen peptides.

8. The topical composition of claim 1, comprising 3%-12% w/w ceramide.

9. The topical composition of claim 1, comprising 1%-5% w/w retinoid.

10. The topical composition of claim 1, wherein hyaluronate and ascorbate are within 5% w/w of each other.

11. A process for preparing a topical product for managing fragile skin:
diluting a topical composition for managing fragile skin with a cosmetic vehicle suitable for topical application to skin, thereby forming a topical product for managing fragile skin, the topical composition being 1%-40% w/w of the topical product, the topical composition, by % w/w of the topical composition, comprising:
a moisturizer component including 15%-25% w/w hyaluronate;
an antioxidant component including 15%-25% w/w ascorbate;
a peptide component including 10%-30% w/w collagen peptides;
a ceramide component including 3%-20% w/w ceramide; and
a retinoid component including 0.1%-10% w/w retinoid;
wherein the % w/w of the collagen peptides is 2.5 to 4 times greater than the % w/w of the ceramide.

12. The process for preparing the topical product for managing fragile skin of claim 11, wherein the cosmetic vehicle is selected from a skin cleanser, skin lotion, skin ointment, skin serum, and a skin cream.

13. The process for preparing the topical product for managing fragile skin of claim 11, wherein the cosmetic vehicle is a skin cleanser and the topical composition is 1%-10% w/w of the topical product.

14. The process for preparing the topical product for managing fragile skin of claim 11, wherein the topical composition comprises 19%-21% w/w hyaluronate.

15. The process for preparing the topical product for managing fragile skin of claim 11, wherein the moisturizer component further includes 1%-13% w/w calendula, 0.5%-6% w/w glycerin, 0.1%-4% w/w lanolin, 0.5%-5% w/w aloe vera, and 0.1%-4% w/w dimethicone.

16. The process for preparing the topical product for managing fragile skin of claim 11, wherein the topical composition comprises 19%-21% w/w ascorbate.

17. The process for preparing the topical product for managing fragile skin of claim 11, wherein the antioxidant component further includes 2%-12% w/w vitamin E, 0.5%-5% w/w niacinamide, 0.5%-5% w/w green tea extract, 0.5%-5% w/w curcumin, and 0.5%-5% w/w zinc.

18. The process for preparing the topical product for managing fragile skin of claim 11, wherein the collagen peptides are primarily composed of proline, glycine, and hydroxyproline as individual amino acids or oligopeptides with 30 or fewer amino acids.

19. The process for preparing of the topical product for managing fragile skin of claim 11, wherein the topical composition comprises 18%-22% w/w collagen peptides.

20. The process for preparing the topical product for managing fragile skin of claim 11, wherein the topical composition comprises 3%-12% w/w ceramide.

21. The process for preparing the topical product for managing fragile skin of claim 11, wherein the topical composition comprises 1%-5% w/w retinoid.

22. The process for preparing the topical product for managing fragile skin of claim 11, wherein hyaluronate and ascorbate are within 5% w/w of each other.

23. A method of managing dermatoporosis, the method comprising:
applying to skin having dermatoporosis a topical product comprising a hyaluronate, an ascorbate, collagen peptides, a ceramide, and a retinoid;
wherein a % w/w of the collagen peptides is 2.5 to 4 times greater than a % w/w of the ceramide.

24. The method of managing dermatoporosis of claim 23, wherein the topical product comprises 0.15%-2.5% w/w hyaluronate, 0.15%-2.5% w/w ascorbate, 0.1%-3% w/w collagen peptides, 0.03%-2% w/w ceramide, and 0.001%-1% retinoid.

25. The method of managing dermatoporosis of claim 23, wherein the topical product comprises 0.15%-10% w/w hyaluronate; 0.15%-10% w/w ascorbate, 0.1%-12% w/w collagen peptides, 0.03%-8% w/w ceramide, and 0.001%-4% retinoid.

26. The method of managing dermatoporosis of claim 23, wherein the topical product comprises 0.15%-2.5% w/w hyaluronate, 0.001%-1.3% w/w calendula, 0.005%-0.6% w/w glycerin, 0.001%-0.4% w/w lanolin, 0.005%-0.5% w/w aloe vera, and 0.001%-0.4% dimethicone.

27. The method of managing dermatoporosis of claim 23, wherein the topical product comprises 0.15%-10% w/w hyaluronate, 0.001%-5.2% w/w calendula, 0.005%-2.4% w/w glycerin, 0.001%-1.6% w/w lanolin, 0.005%-2% w/w aloe vera, and 0.001%-1.6% dimethicone.

28. The method of managing dermatoporosis of claim 23, wherein the topical product comprises 0.15%-2.5% w/w hyaluronate, 0.15%-2.5% w/w ascorbate, 0.2%-1.2% w/w vitamin E, 0.005%-0.5% w/w niacinamide, 0.005%-0.5% w/w green tea extract, 0.005%-0.5% w/w curcumin, and 0.005%-0.5% w/w zinc.

29. The method of managing dermatoporosis of claim 23, wherein the topical product comprises 0.15%-10% w/w hyaluronate, 0.15%-10% w/w ascorbate, 0.2%-4.8% w/w vitamin E, 0.005%-2% w/w niacinamide, 0.005%-2% w/w green tea extract, 0.005%-2% w/w curcumin, and 0.005%-2% w/w zinc.

30. The method of managing dermatoporosis of claim 23, wherein the collagen peptides are primarily composed of proline, glycine, and hydroxyproline as individual amino acids or oligopeptides with 30 or fewer amino acids.

31. The method of managing dermatoporosis of claim 23, wherein the topical product comprises 0.1%-3% w/w collagen peptides.

32. The method of managing dermatoporosis of claim 23, wherein the topical product comprises 0.1%-12% w/w collagen peptides.

33. The method of managing dermatoporosis of claim 23, wherein the topical product comprises 0.03%-2% w/w ceramide.

34. The method of managing dermatoporosis of claim 23, wherein the topical product comprises 0.03%-8% w/w ceramide.

35. The method of managing dermatoporosis of claim 23, wherein the topical product comprises 0.01%-1% w/w retinoid.

36. The method of managing dermatoporosis of claim 23, wherein the topical product comprises 0.01%-4% w/w retinoid.

37. The method of managing dermatoporosis of claim 23, wherein hyaluronate and ascorbate are within 5% w/w of each other.

38. A topical product for managing fragile skin, the topical product comprising:
a moisturizer component including 0.15%-10% w/w hyaluronate;
an antioxidant component including 0.15%-10% w/w ascorbate;
a peptide component including 0.1%-12% w/w collagen peptides;
a ceramide component including 0.03%-8% w/w ceramide; and
a retinoid component including 0.001%-4% w/w retinoid;
wherein the % w/w of the collagen peptides is 2.5 to 4 times greater than the % w/w ceramide.

39. The topical product of claim 38, comprising 0.15%-2.5% w/w hyaluronate, 0.15%-2.5% w/w ascorbate, 0.1%-3% w/w collagen peptides, 0.03%-2% w/w ceramide, and 0.001%-1% retinoid.

40. The topical product of claim 38, comprising 0.15%-2.5% w/w hyaluronate, 0.001%-1.3% w/w calendula, 0.005%-0.6% w/w glycerin, 0.001%-0.4% w/w lanolin, 0.005%-0.5% w/w aloe vera, and 0.001%-0.4% dimethicone.

41. The topical product of claim 38, comprising 0.001%-5.2% w/w calendula, 0.005%-2.4% w/w glycerin, 0.001%-1.6% w/w lanolin, 0.005%-2% w/w aloe vera, and 0.001%-1.6% dimethicone.

42. The topical product of claim 38, comprising 0.15%-2.5% w/w hyaluronate, 0.15%-2.5% w/w ascorbate, 0.2%-1.2% w/w vitamin E, 0.005%-0.5% w/w niacinamide, 0.005%-0.5% w/w green tea extract, 0.005%-0.5% w/w curcumin, and 0.005%-0.5% w/w zinc.

43. The topical product of claim 38, comprising 0.15%-10% w/w ascorbate, 0.2%-4.8% w/w vitamin E, 0.005%-2% w/w niacinamide, 0.005%-2% w/w green tea extract, 0.005%-2% w/w curcumin, and 0.005%-2% w/w zinc.

44. The topical product of claim 38, wherein the collagen peptides are primarily composed of proline, glycine, and hydroxyproline as individual amino acids or oligopeptides with 30 or fewer amino acids.

45. The topical product of claim 38, comprising 0.1%-3% w/w collagen peptides.

46. The topical product of claim 38, comprising 0.1%-12% w/w collagen peptides.

47. The topical product of claim 38, comprising 0.03%-2% w/w ceramide.

48. The topical product of claim 38, comprising 0.03%-8% w/w ceramide.

49. The topical product of claim 38, comprising 0.01%-1% w/w retinoid.

50. The topical product of claim 38, comprising 0.01%-4% w/w retinoid.

51. The topical product of claim 38, wherein hyaluronate and ascorbate are within 5% w/w of each other.
